(19) United States
(12) Patent Application Publication
(10) Pub. No.: US 2011/0009416 A1
(43) Pub. Date: Jan. 13, 2011

(54) PH INDEPENDENT FORMULATIONS OF 6-(5-CHLORO-2-PYRIDYL)-5-[(4-METHYL-1-PIPERAZINYL)CARBONYLOXY]-7-OXO-6,7-DIHYDRO-5H-PYRROLO[3,4-b]PYRAZINE

(75) Inventors: Richard HSIA, Boston, MA (US); Tushar MISRA, Norfolk, MA (US); Priya CAPILA, Ashland, MA (US); Ge BAI, Marlborough, MA (US)

Correspondence Address: HESLIN ROTHENBERG FARLEY & MESITI P.C., 5 COLUMBIA CIRCLE, ALBANY, NY, 12203, US

(73) Assignee: SEPRACOR INC., Marlborough, MA (US)

(21) Appl. No.: 12/498,751

(22) Filed: Jul. 7, 2009

(51) Int. Cl. A61K 31/4985; A61P 1/00

(52) U.S. Cl. 514/249

(57) ABSTRACT

Pharmaceutical compositions of zopiclone [(6-(5-chloro-2-pyridyl)-5-[(4-methyl-1-piperazinyl)carbonyloxy]-7-oxo-6,7-dihydro-5H-pyrrolo[3,4-b]pyrazine)] that render the aqueous solubility/dissolution of the free base independent of the pH of the gastrointestinal tract are disclosed. The compositions are useful for oral administration.

### CROSS REFERENCE TO RELATED APPLICATIONS

This application is filed contemporaneously with a PCT application entitled, “pH INDEPENDENT FORMULATIONS OF 6-(5-CHLORO-2-PYRIDYL)-5-[(4-METHYL-1-PIPERAZINYL)CARBONYLOXY]-7-OXO-6,7-DIHYDRO-5H-PYRROLO[3,4-b]PYRAZINE.” No serial number has been generated for this PCT filing as yet.

### FIELD OF THE INVENTION

The present invention relates to pharmaceutical compositions of (6-(5-chloro-2-pyridyl)-5-[(4-methyl-1-piperazinyl)carbonyloxy]-7-oxo-6,7-dihydro-5H-pyrrolo[3,4-b]pyrazine) that render the aqueous solubility/dissolution of the free base independent of the pH of the gastrointestinal tract. The compositions are useful for oral administration.

### BACKGROUND OF THE INVENTION

Eszopiclone, also known as (S)-zopiclone or (S)-(6-(5-chloro-2-pyridyl)-5-[(4-methyl-1-piperazinyl)carbonyloxy]-7-oxo-6,7-dihydro-5H-pyrrolo[3,4-b]pyrazine), is formulated as the free base and is sold as LUNESTA®. It is used to treat different types of sleep problems, such as difficulty in falling asleep, difficulty in maintaining sleep during the night, and waking up too early in the morning. Most people with insomnia have more than one of these problems. See, e.g., WO 93/10787; Brun, J. P., Pharm. Biochem. Behav. 29: 831 832 (1988).

The compound eszopiclone and various methods of treatment are disclosed at least in the following U.S. Pat. Nos. 7,125,874; 6,864,257; 6,444,673; 6,319,926; and 5,786,357.

Racemic zopiclone, rac-(6-(5-chloro-2-pyridyl)-5-[(4-methyl-1-piperazinyl)carbonyloxy]-7-oxo-6,7-dihydro-5H-pyrrolo[3,4-b]pyrazine), also formulated as the free base, has been sold in Europe for many years to treat different types of sleep problems.

### SUMMARY OF THE INVENTION

In one aspect, the present invention relates to an oral pharmaceutical dosage form comprising: (a) eszopiclone free base; and (b) a super-stoichiometric amount of a solid acid.

In another aspect, the invention relates to methods for (1) rendering the dissolution of orally administered racemic or enantioenriched zopiclone pH independent; (2) accelerating the postprandial dissolution of orally administered racemic or enantioenriched zopiclone; and (3) improving the bioavailability of orally administered racemic or enantioenriched zopiclone. The methods comprise orally administering racemic or enantioenriched zopiclone free base in the presence of a stoichiometric excess of an acid chosen from those disclosed above. Alternatively, the methods comprise orally administering a dosage form as described above.

### BRIEF DESCRIPTION OF THE DRAWINGS

FIG. 1 depicts exemplary dissolution profiles of the eszopiclone free base compounded with 9% fumaric acid at varying pH.

FIG. 2 depicts exemplary dissolution profiles of the eszopiclone free base compounded with various acids compared to free base in water.

FIG. 3 depicts exemplary dissolution profiles of the eszopiclone free base and eszopiclone compounded with 9% fumaric acid at pH 1.

FIG. 4 depicts exemplary dissolution profiles of the eszopiclone free base and eszopiclone compounded with 1% fumaric acid, 3% fumaric acid, 9% fumaric acid, and 20% fumaric acid at pH 6.8 in water.

### DETAILED DESCRIPTION

The invention relates to oral dosage forms of the free base of racemic or enantioenriched zopiclone. It has surprisingly been found that these free-base forms can be formulated with a stoichiometric excess of a solid acid, particularly an acid chosen from adipic acid, aspartic acid, glucoheptonic acid, gluconic acid, glutamic acid, lactic acid, mandelic acid, malic acid, maleic acid, fumaric acid, citric acid, ascorbic acid, phosphoric acid, tartaric acid, toluenesulfonic acid, benzenesulfonic acid, succinic acid, monosodium phosphate, and glucuronic acid. The resulting oral solid dosage form will exhibit a dissolution profile that is independent of the pH of the dissolving medium. The term “solid acid” refers to an acid, for example a carboxylic acid, a sulfonic acid or a phosphoric acid, that is a solid at 25° C. The term “super-stoichiometric amount” refers to an amount of the acid that exceeds a 1:1 molar ratio of acid to the free base. Thus a salt of zopiclone with a monobasic acid, for example a benzenesulfonic acid salt, would contain a 1:1 molar ratio of zopiclone to benzenesulfonic acid, and a super-stoichiometric amount would be an amount greater than one molar equivalent of benzenesulfonic acid. A salt of zopiclone with a dibasic acid, for example a malic acid salt, could contain a 2:1 or a 1:1 molar ratio of zopiclone to malic acid; nonetheless, a super-stoichiometric amount for the purpose of the present invention would be an amount greater than one molar equivalent of malic acid. A salt with a tribasic acid, for example a citric acid salt, could contain a 3:1, 2:1 or a 1:1 molar ratio of zopiclone to citric acid; a super-stoichiometric amount for the purpose of the present invention would still be an amount greater than one molar equivalent of citric acid. The acid can be present in an amount between about 0.1%, 0.5%, 1%, 2% or 3% by weight—on the low end of the range—and about 10%, 15%, 20%, 25%, and 30% by weight—on the high end of the range—based on the total weight of the dosage form.

In some embodiments the acid is present in an amount between about 0.5% by weight and about 30% by weight of the dosage form. In some embodiments the acid is present in an amount between about 1% by weight and about 20% by weight of the dosage form. In some embodiments the acid is present in an amount between about 2% by weight and about 10% by weight of the dosage form.

In one embodiment, the acid is chosen from malic acid, maleic acid, fumaric acid, citric acid, ascorbic acid, phosphoric acid, tartaric acid and monosodium phosphate. In one embodiment, the acid can be maleic acid; in another embodiment, the acid can be fumaric acid; in another embodiment, the acid can be L-malic acid; and in another embodiment, the acid can be D-malic acid.

In various embodiments, the racemic or enantioenriched zopiclone can be present in amounts of 0.5 mg, 1 mg, 1.5 mg, 2 mg, 2.5 mg, 3 mg, 4 mg and 5 mg or fractional increments of 0.1 mg in between. A particular embodiment is a 100 mg tablet comprising an amount between about 1 mg to about 3 mg of eszopiclone and an amount between about 3 to about 9 mg of acid.

The invention is also directed towards a method for treating a sleep disorder in a subject comprising administering to a subject in need thereof a composition as described above. The invention provides a method for treating and/or preventing sleep disorders, including primary insomnia and sleep-awake rhythm disorders (e.g., work-shift syndrome, time-zone syndrome (jet-lag)). Insomnia is characterized by difficulty in sleeping or disturbed sleep patterns. Insomnia can be of a primary nature with little apparent relationship to immediate somatic or psychic events, or secondary to some acquired pain, anxiety or depression.

In another aspect, the invention provides a method for treating anxiety in a subject comprising administering to a subject in need thereof a composition as described above. As used herein the term “anxiety” refers to an anxiety disorder. Examples of anxiety disorders treatable by the compositions and methods disclosed herein include, but are not limited to: panic attack, agoraphobia, acute stress disorder, specific phobia, panic disorder, psychoactive substance anxiety disorder, organic anxiety disorder, obsessive-compulsive anxiety disorder, posttraumatic stress disorder and generalized anxiety disorder. Anxiety as referred to herein also includes situational anxiety (e.g. as experienced by a performer prior to a performance). The named anxiety disorders have been characterized in the DSM-IV-R. Diagnostic and Statistical Manual of Mental Disorders, Revised, 4th Ed. (1994). The DSM-IV-R was prepared by the Task Force on Nomenclature and Statistics of the American Psychiatric Association, and provides clear descriptions of diagnostic categories.

As used herein, the terms zopiclone and 6-(5-chloro-2-pyridyl)-5-[(4-methyl-1-piperazinyl)carbonyloxy]-7-oxo-6,7-dihydro-5H-pyrrolo[3,4-b]pyrazine refer to compounds represented by the following structure:

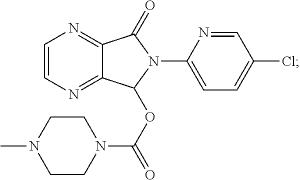

the terms eszopiclone, LUNESTA®, and [(9S)-8-(5-chloropyridin-2-yl)-7-oxo-2,5,8-triazabicyclo[4.3.0]nona-1,3,5-trien-9-yl]-4-methyl piperazine-1-carboxylate refer to an individual enantiomer of the foregoing represented by the following structure:

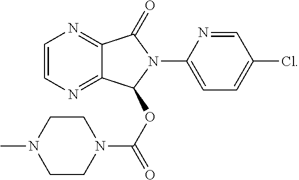

The compositions and methods of the invention contemplate both the racemic mixture (also known as zopiclone), and in certain embodiments, contemplates a single enantiomer, e.g., the S-enantiomer (eszopiclone). Eszopiclone is the S-(+)-optical isomer of the compound zopiclone, which is described in U.S. Pat. Nos. 6,319,926 and 6,444,673. This isomer, which will hereinafter be referred to by its USAN-approved generic name, eszopiclone, includes the optically pure and the substantially optically pure (e.g., 90%, 95% or 99% optical purity) S-(+)-zopiclone isomer. Accordingly, the compositions of the present invention will include not only a stereoisomeric mixture, but also individual respective stereoisomers substantially free from other stereoisomers. In certain embodiments, it encompasses non-racemic mixtures of stereoisomers of the same compound (e.g., about 90, 80, 70, or 60 weight percent of one enantiomer and about 10, 20, 30, or 40 weight percent of the opposite enantiomer); and mixtures of different racemic or stereomerically pure compounds (e.g., about 90, 80, 70, or 60 weight percent of one compound and about 10, 20, 30, or 40 weight percent of another). As used herein, the term “enantioenriched” refers to a sample of a chiral compound that consists of more of one enantiomer than the other. The extent to which a sample is enantiomerically enriched is quantitated by the enantiomeric purity or the enantiomeric excess. For example, the term “enantioenriched zopiclone” is intended to refer to a sample in which there is more (S)-zopiclone (eszopiclone) than (R)-zopiclone. In some embodiments, “enantioenriched zopiclone” refers to eszopiclone, which is substantially free of (R)-zopiclone. For example, “enantioenriched zopiclone” is intended to refer to eszopiclone in which the enantiomeric purity is not more than about 15% (R)-zopiclone, or not more than about 10% (R)-zopiclone, or not more than about 5% (R)-zopiclone, or preferably not more than about 2% (R)-zopiclone, or more preferably not more than about 1% (R)-zopiclone.

The compounds of the invention can be synthesized by techniques known in the art. The starting materials and certain intermediates used in the synthesis of the compounds of this invention are available from commercial sources or can themselves be synthesized using reagents and techniques known in the art, including those synthesis schemes delineated herein.

Racemic zopiclone is commercially available and can be made using various methods, such as those disclosed in U.S. Pat. Nos. 3,862,149 and 4,220,646.

The term “treating” or “treated” refers to administering a compound described herein to a subject with the purpose to cure, heal, alleviate, relieve, alter, remedy, ameliorate, improve, or affect a disease, the symptoms of the disease or the predisposition toward the disease.

An “effective amount” refers to an amount of a compound, which confers a therapeutic effect on the treated subject. The therapeutic effect can be objective (i.e., measurable by some test or marker) or subjective (i.e., subject gives an indication of or feels an effect). Effective doses will also vary depending on route of administration.

As used herein, and unless otherwise specified, the terms “prevent,” “preventing,” “prevention,” and “prophylactic” refer to the prevention of the onset, recurrence or intensification of a disorder disclosed herein. The terms “prevent,” “preventing,” “prevention,” and “prophylactic” include ameliorating and/or reducing the occurrence of symptoms of a disorder disclosed herein.

The present invention relates to pharmaceutical compositions containing the free base of racemic or enantioenriched zopiclone and a super-stoichiometric amount of an acid chosen from: adipic acid, aspartic acid, glucoheptonic acid, gluconic acid, glutamic acid, lactic acid, mandelic acid, malic acid, maleic acid, fumaric acid, citric acid, ascorbic acid, phosphoric acid, tartaric acid, toluenesulfonic acid, benzenesulfonic acid, succinic acid, monosodium phosphate and glucuronic acid.

Pharmaceutical compositions and dosage forms of the invention comprise one or more of the active ingredients disclosed herein. Pharmaceutical compositions and dosage forms of the invention typically also comprise one or more pharmaceutically acceptable excipients or diluents.

The term “pharmaceutically acceptable,” as used herein, refers to a component that is, within the scope of sound medical judgment, suitable for use in contact with the tissues of humans and other mammals without undue toxicity, irritation, allergic response and the like, and is commensurate with a reasonable benefit/risk ratio. Preferably, a composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that can be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

Single unit dosage forms of the invention are suitable for oral administration to a patient. Examples of dosage forms include, but are not limited to: tablets; caplets; capsules, such as soft elastic gelatin capsules; cachets; troches; lozenges and dispersions.

The composition, shape, and type of dosage forms of the invention will typically vary depending on their use. For example, a dosage form used in the acute treatment of a disorder can contain larger amounts of one or more of the active ingredients it comprises than a dosage form used in the chronic treatment of the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets can contain excipients not suited for use in parenteral dosage forms. The suitability of a particular excipient can also depend on the specific active ingredients in the dosage form. For example, the decomposition of some active ingredients can be accelerated by some excipients such as lactose, or when exposed to water. This invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art. In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379 80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid. In this regard, ascorbic acid can be present either as an antioxidant in a composition with another acid described above, or it can function on its own as both the super-stoichiometric acid and the antioxidant.

Typical oral dosage forms of the invention are prepared by combining the active ingredient(s) in an intimate admixture with at least one excipient according to conventional pharmaceutical compounding techniques. Excipients suitable for use in oral dosage forms include, but are not limited to, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent. Tablets can be prepared using any conventional pharmaceutical solid dosage unit operations, such as dry blending, wet or dry granulations, milling, compression, pelletizing and coating.

In dry blending and direct compression approach, the active (eszopiclone) is mixed with acidulants and other excipient in serial dilution. An example of dry blending and direct compression is provided in the steps outlined below: add approximate half portion of microcrystalline cellulose and eszopiclone and acidulants to a mixer and mix; add the remainder of microcrystalline cellulose, colloidal silicon dioxide and croscarmellose sodium to the mixture from above and mix; add dibasic calcium phosphate anhydrous to the mixture from above and mix; add magnesium stearate to the mixture from above and mix; and compress the mixture into tablets using a rotary tablet press.

In some embodiments, wet granulation methods can be used to form the solid dosage unit. Below is an example of wet granulation method that can be used with the present invention. Wet granulation approach binder is added to a pre-mixture that contains eszopiclone. The resulted wet granules containing eszopiclone and acidulants is dried using, for example, a tray dryer or fluid bed dryer. The dried granules are compressed into tablets or filled into capsules. The binder can be a solvent such as water, or alcohol, or the mixture of water and alcohol. Polymers, such as starch, can be added to the solvent as part of the binder. The acidulants can be added to the solvent as part of the binder. The pre-mixture can be eszopiclone and acidulants. The pre-mixture can consist of eszopiclone, acidulants and other excipient. The pre-mixture may consist of eszopiclone and other excipient when acidulants was incorporated totally in the binder. If control of particle size distribution is desired, the dried granules can be milled. The wet granulation approach may be achieved by high shear granulation equipments or fluid bed technology or spray during technology.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-β-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

Zopiclone can, for example, be administered with a dosage ranging from about 0.001 to about 0.2 mg/kg of body weight, alternatively dosages between 0.1 mg and 15 mg/dose, or between about 0.1 mg and about 10 mg/dose, or preferably between about 0.2 mg and about 5 mg/dose, or according to the requirements of the particular therapy. The methods herein contemplate administration of an effective amount of compound or compound composition to achieve the desired or stated effect. Typically, the pharmaceutical compositions of this invention will be administered from about 1 to about 6 times per day. Such administration can be used as a chronic or acute therapy. The amount of active ingredient that can be combined with the carrier materials to produce a single dosage form will vary depending upon the host treated and the particular mode of administration. In some embodiments, such preparations contain from about 20% to about 80% (w/w) active compound. In some embodiments, such preparations contain from about 0.5% to about 20% active compound. A typical preparation will contain from about 0.5% to about 5% active compound (w/w).

Lower or higher doses than those recited above can be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Like the amounts and types of excipients, the amounts and specific types of active ingredients in a dosage form can differ depending on factors such as, but not limited to, the route by which it is to be administered to patients. In human therapy, the doses depend on the effect sought and the treatment period; taken orally, they are generally between 0.5 and 15 mg per day for an adult. For many applications, unit dosages containing 0.5 mg, 1 mg, 2 mg or 3 mg of a present eszopiclone salt will be suitable. In some embodiments, the unit dosages of the eszopiclone salts can be adjusted to contain the molar equivalent of 0.5 mg, 1 mg, 2 mg or 3 mg of eszopiclone freebase.

One aspect of the present invention relates to combination therapy. This type of therapy is advantageous because the co-administration of active ingredients achieves a therapeutic effect that is greater than the therapeutic effect achieved by administration of only a single therapeutic agent. In one embodiment, the co-administration of two or more therapeutic agents achieves a synergistic effect, i.e., a therapeutic affect that is greater than the sum of the therapeutic effects of the individual components of the combination. In another embodiment, the co-administration of two or more therapeutic agents achieves an augmentation effect. The active ingredients that comprise a combination therapy can be administered together via a single dosage form. The agents can be formulated into a single tablet, pill, capsule, and the like.

The dosage of the active agents will generally be dependent upon a number of factors including pharmacodynamic characteristics of each agent of the combination, mode and route of administration of active agent(s), the health of the patient being treated, the extent of treatment desired, the nature and kind of concurrent therapy, if any, and the frequency of treatment and the nature of the effect desired. In general, dosage ranges of the active agents often range from about 0.001 to about 250 mg/kg body weight per day. For example, for a normal adult having a body weight of about 70 kg, a dosage in the range of from about 0.1 to about 25 mg/kg body weight is typically preferred. However, some variability in this general dosage range may be required depending upon the age and weight of the subject being treated, the particular agent being administered and the like. Since two or more different active agents are being used together in a combination therapy, the potency of each agent and the interactive effects achieved using them together must be considered. Importantly, the determination of dosage ranges and optimal dosages for a particular mammal is also well within the ability of one of ordinary skill in the art having the benefit of the instant disclosure.

In certain embodiments, it can be advantageous for the pharmaceutical combination to have a relatively large amount of the first component compared to the second component. In certain instances, the ratio of the first active agent to second active agent is 30:1, 20:1, 15:1, 10:1, 9:1, 8:1, 7:1, 6:1, or 5:1. In certain embodiments, it can be preferable to have a more equal distribution of pharmaceutical agents. In certain instances, the ratio of the first active agent to the second active agent is 4:1, 3:1, 2:1, 1:1, 1:2, 1:3, or 1:4. In certain embodiments, it can be advantageous for the pharmaceutical combination to have a relatively large amount of the second component compared to the first component. In certain instances, the ratio of the second active agent to the first active agent is 30:1, 20:1, 15:1, 10:1, 9:1, 8:1, 7:1, 6:1, or 5:1.

For example, a formulation intended for oral administration to humans may contain from 0.1 mg to 5 g of the first therapeutic agent and 0.1 mg to 5 g of the second therapeutic agent, both of which are compounded with an appropriate and convenient amount of carrier material varying from about 5 to about 95 percent of the total composition. Unit dosages will generally contain between from about 0.5 mg to about 1500 mg of the first therapeutic agent and 0.5 mg to about 1500 mg of the second therapeutic agent. In a preferred embodiment, the dosage comprises 0.5 mg, 1 mg, 2 mg, 3 mg, 4 mg, 5 mg, 10 mg, 25 mg, 50 mg, 100 mg, 200 mg, 300 mg, 400 mg, 500 mg, 600 mg, 800 mg, or 1000 mg, etc., up to 1500 mg of the first therapeutic agent. In a preferred embodiment, the dosage comprises 0.5 mg, 1 mg, 2 mg, 3 mg, 4 mg, 5 mg, 10 mg, 25 mg, 50 mg, 100 mg, 200 mg, 300 mg, 400 mg, 500 mg, 600 mg, 800 mg, or 1000 mg, etc., up to 1500 mg of the second therapeutic agent. The optimal ratios of the first and second therapeutic agent can be determined by standard assays known in the art.

In one embodiment, the present invention relates to a dosage form comprising a pharmaceutical composition of the present invention, and one or more antidepressant. Nonlimiting examples of antidepressants include without limitation selective serotonin reuptake inhibitors, serotonin reuptake inhibitors, norepinephrine reuptake inhibitors, dopamine reuptake inhibitors, 5-HT_{2A} receptor modulators, triple reuptake inhibitors, and double reuptake inhibitors. In another aspect, the present invention discloses a method of treating a patient suffering from a mood disorder, comprising the step of co-administering to a patient in need thereof a therapeutically effective amount of a composition of the present invention, and an antidepressant. Nonlimiting examples of 5-HT_{2A} receptor modulators include MDL 100907, SR 46349B, YM 992, fananserin, oxazolidine compounds A, phenylindole compounds A, piperidinyl compounds B, spiroazacyclic compounds C, or azacyclic compounds D, or a pharmaceutically acceptable salt, clathrate, polymorph, or co-crystal of any one of them. Nonlimiting examples of serotonin reuptake inhibitors include citalopram, duloxetine, escitalopram, fluoxetine, fluvoxamine, milnacipran, paroxetine, sertraline, clominpramine, femoxetine, indapline, alaprolclate, cericlamine, or ifoxetine, or a pharmaceutically acceptable salt, clathrate, polymorph, or co-crystal of any one of them. Nonlimiting examples of norepinephrine reuptake inhibitors include desipramine, maprotiline, lofepramine, reboxetine, oxaprotiline, fezolamine, tomoxetine, or (S,S)-hydroxybupropion, or a pharmaceutically acceptable salt, clathrate, polymorph, or co-crystal of any one of them.

The following non-limiting examples are illustrative of the invention.

Dry blending and direct compression preparation methods were used for the preparation of following formulation examples.

### Example 1

| | |
|---|---|
| Eszopiclone | 3 mg |
| Fumaric acid | 1 mg |
| MCC | 63.3 mg |
| A-Tab | 30 mg |
| SiO2 | 0.2 mg |
| Croscarmellose | 2 mg |
| Mg. Stearate | 0.5 mg |

### Example 2

| | |
|---|---|
| Eszopiclone | 3 mg |
| Fumaric acid | 3 mg |
| MCC | 61.3 mg |
| A-Tab | 30 mg |
| SiO2 | 0.2 mg |
| Croscarmellose | 2 mg |
| Mg. Stearate | 0.5 mg |

### Example 3

| | |
|---|---|
| Eszopiclone | 3 mg |
| Fumaric acid | 9 mg |
| MCC | 55.3 mg |
| A-Tab | 30 mg |
| SiO2 | 0.2 mg |
| Croscarmellose | 2 mg |
| Mg. Stearate | 0.5 mg |

### Example 4

| | |
|---|---|
| Eszopiclone | 3 mg |
| Fumaric acid | 18 mg |
| MCC | 46.3 mg |
| A-Tab | 30 mg |
| SiO2 | 0.2 mg |
| Croscarmellose | 2 mg |
| Mg. Stearate | 0.5 mg |

### Example 5

| | |
|---|---|
| Eszopiclone | 3 mg |
| L-malic acid | 3 mg |
| MCC | 61.3 mg |
| A-Tab | 30 mg |
| SiO2 | 0.2 mg |
| Croscarmellose | 2 mg |
| Mg. Stearate | 0.5 mg |

### Example 6

| | |
|---|---|
| Eszopiclone | 3 mg |
| Maleic acid | 9 mg |
| MCC | 55.3 mg |
| A-Tab | 30 mg |
| SiO2 | 0.2 mg |
| Croscarmellose | 2 mg |
| Mg. Stearate | 0.5 mg |

### Example 7

| | |
|---|---|
| Eszopiclone | 3 mg |
| MCC | 64.3 mg |
| A-Tab | 30 mg |
| SiO2 | 0.2 mg |
| Croscarmellose | 2 mg |
| Mg. Stearate | 0.5 mg |

In examples 2 to 4, the dissolution in water increased significantly as the fumaric acid was increased up to about 9%. Addition of acid beyond about 9% did not further affect dissolution in water. L-malic had a similar affect on the dissolution as fumaric acid. At the same weight percent, for example 3% L-malic acid seemed to make dissolution in water slightly faster than that of fumaric acid. The addition of maleic acid at 9% had a similar affect to that of 9% fumaric acid on dissolution in water.

Seven batches of 3 mg eszopiclone core tablets were prepared with three different acids at different concentrations. The acids used were fumaric acid (1%, 3%, 9% and 20%), L-malic acid (3% and 12%) and maleic acid (9%). All core tablets were made by dry blend and direct compression method. Formulations:

| 1% Fumaric Acid | | Amount (g) |
|---|---|---|
| 1 | Eszopiclone (thru' #80 mesh) | 30 |
| 2 | Fumaric Acid | 10 |
| 3 | Avicel pH 102 (Microcrystalline Cellulose) | 502.2 |
| 4 | A-Tab (Dicalcium Phosphate, Anhydrous) | 420 |
| 5 | Ac-Di-Sol (Croscarmellose Sodium) | 28 |
| 6 | Colloidal Silica (Hydrophilic Pyrogenic Silica) | 2.8 |
| 7 | Magnesium Stearate | 7 |
| | Batch Size | 1000 |
| 3% Fumaric Acid | | Amount (g) |
| 1 | Eszopiclone (thru' #80 mesh) | 30 |
| 2 | Fumaric Acid | 30 |
| 3 | Avicel pH 102 (Microcrystalline Cellulose) | 482.2 |
| 4 | A-Tab (Dicalcium Phosphate, Anhydrous) | 420 |
| 5 | Ac-Di-Sol (Croscarmellose Sodium) | 28 |
| 6 | Colloidal Silica (Hydrophilic Pyrogenic Silica) | 2.8 |
| 7 | Magnesium Stearate | 7 |
| | Batch Size | 1000 |
| 9% Fumaric Acid | | Amount (g) |
| 1 | Eszopiclone (thru' #80 mesh) | 30 |
| 2 | Fumaric Acid | 90 |
| 3 | Avicel pH 102 (Microcrystalline Cellulose) | 422.2 |
| 4 | A-Tab (Dicalcium Phosphate, Anhydrous) | 420 |
| 5 | Ac-Di-Sol (Croscarmellose Sodium) | 28 |
| 6 | Colloidal Silica (Hydrophilic Pyrogenic Silica) | 2.8 |
| 7 | Magnesium Stearate | 7 |
| | Batch Size | 1000 |
| 20% Fumaric Acid | | Amount (g) |
| 1 | Eszopiclone (thru' #80 mesh) | 30 |
| 2 | Fumaric Acid | 200 |
| 3 | Avicel pH 102 (Microcrystalline Cellulose) | 312.2 |
| 4 | A-Tab (Dicalcium Phosphate, Anhydrous) | 420 |
| 5 | Ac-Di-Sol (Croscarmellose Sodium) | 28 |
| 6 | Colloidal Silica (Hydrophilic Pyrogenic Silica) | 2.8 |
| 7 | Magnesium Stearate | 7 |
| | Batch Size | 1000 |
| 12% L-Malic | | Amount (g) |
| 1 | Eszopiclone (thru' #80 mesh) | 30 |
| 2 | L-Malic Acid | 120 |
| 3 | Avicel pH 102 (Microcrystalline Cellulose) | 392.2 |
| 4 | A-Tab (Dicalcium Phosphate, Anhydrous) | 420 |
| 5 | Ac-Di-Sol (Croscarmellose Sodium) | 28 |
| 6 | Colloidal Silica (Hydrophilic Pyrogenic Silica) | 2.8 |
| 7 | Magnesium Stearate | 7 |
| | Batch Size | 1000 |
| 3% L-Malic Acid | | Amount (g) |
| 1 | Eszopiclone (thru' #80 mesh) | 30 |
| 2 | L-Malic Acid | 30 |
| 3 | Avicel pH 102 (Microcrystalline Cellulose) | 482.2 |
| 4 | A-Tab (Dicalcium Phosphate, Anhydrous) | 420 |
| 5 | Ac-Di-Sol (Croscarmellose Sodium) | 28 |
| 6 | Colloidal Silica (Hydrophilic Pyrogenic Silica) | 2.8 |
| 7 | Magnesium Stearate | 7 |
| | Batch Size | 1000 |
| 9% Maleic Acid | | Amount (g) |
| 1 | Eszopiclone (thru' #80 mesh) | 30 |
| 2 | Maleic Acid | 90 |
| 3 | Avicel pH 102 (Microcrystalline Cellulose) | 422.2 |
| 4 | A-Tab (Dicalcium Phosphate, Anhydrous) | 420 |
| 5 | Ac-Di-Sol (Croscarmellose Sodium) | 28 |
| 6 | Colloidal Silica (Hydrophilic Pyrogenic Silica) | 2.8 |
| 7 | Magnesium Stearate | 7 |
| | Batch Size | 1000 |

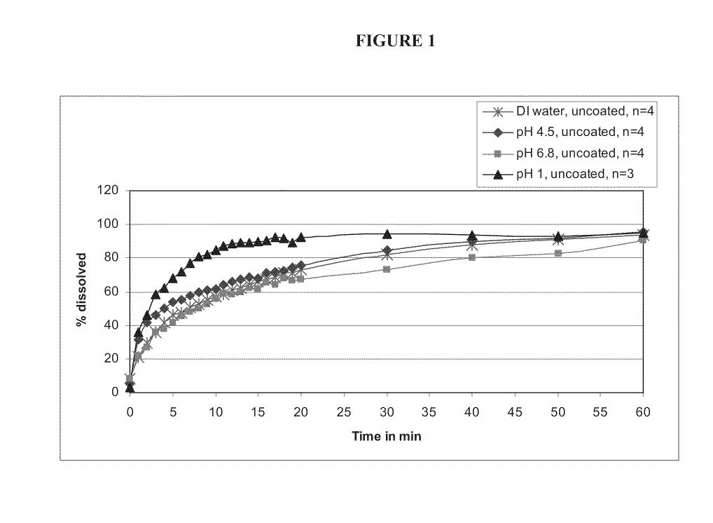

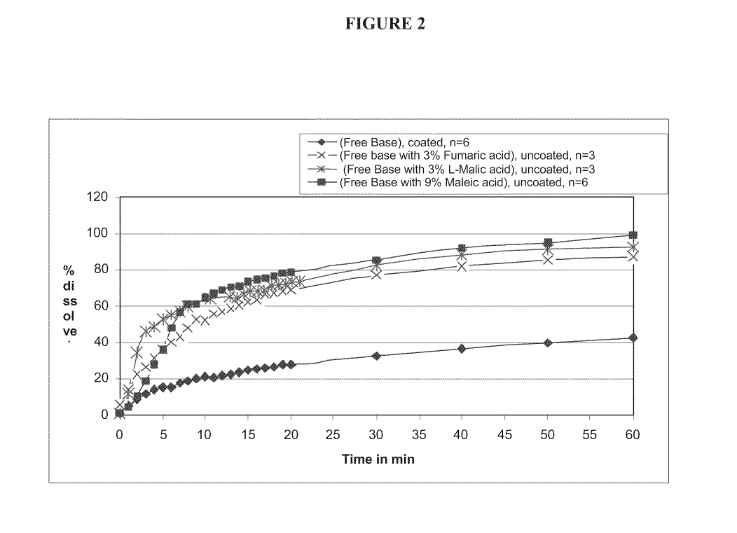

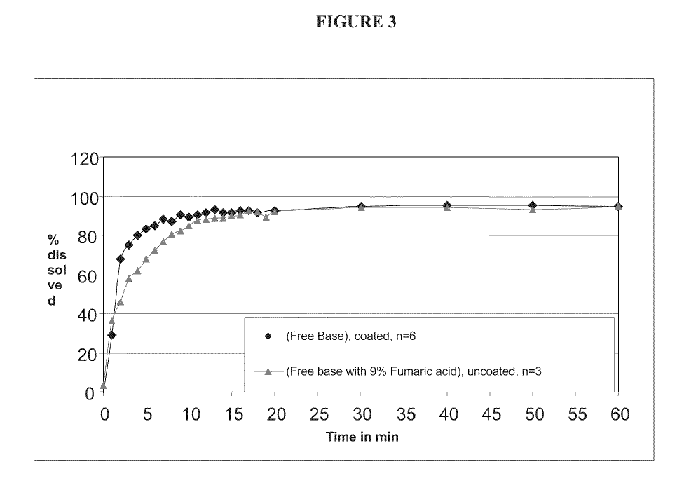

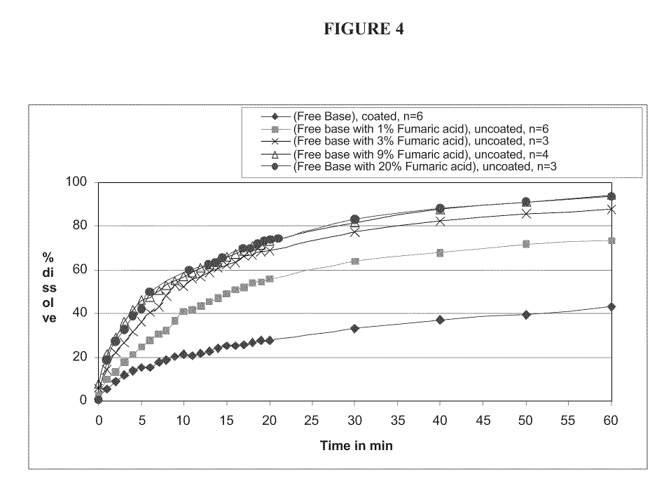

The tablets were tested for solubility according to protocol below. The results are provided in tabular format and in four figures (FIG. 1, FIG. 2, FIG. 3 and FIG. 4.) FIG. 1 depicts the dissolution profiles of the eszopiclone free base compounded with 9% fumaric acid at pH 1, 4.5, 6.8 and in deionized water. It can be seen that the dissolution is relatively independent of pH. FIG. 2 depicts dissolution profiles, in water, of the eszopiclone free base and the free base compounded with 3% fumaric acid, 3% L-malic acid and 9% maleic acid compared to free base. It can be seen that the dissolution is rapid and complete with the compositions of the invention and is slow and incomplete (at one hour) for the free base. FIG. 3 depicts dissolution profiles of the eszopiclone free base and eszopiclone compounded with 9% fumaric acid at pH 1. At pH 1 both compositions dissolve rapidly and completely. FIG. 4 depicts dissolution profiles of the eszopiclone free base and eszopiclone compounded with 1% fumaric acid, 3% fumaric acid, 9% fumaric acid, and 20% fumaric acid in water. The composition of the invention provides rapid and complete dissolution; the free base remains less than 50% dissolved at one hour. The invention can be used to adjust the release rate or dissolution rate of the active ingredient from the tablet. For example, if rapid complete release is desired, the oral dosage form can include greater than about 1% solid acid, or more preferably greater than about 3% solid acid.

Dissolution over time of eszopiclone free base compounded with various solid acids was compared to that of free base at body temperature (37° C.) in dissolution media at various pH values using Hanson Research SR8—Plus Dissolution Apparatus and C-Technologies Fiber Optic UV Probes at 305 nm (excipients subtracted at 410 nm). The dissolution media used were (a) pH 1 (0.1N HCl) 50 ml of conc HCl mixed in 6 L of water; (b) pH 4.5 acetate buffer (20 mM) 5.88 g of sodium acetate trihydrate dissolved in 6 L of water, pH adjusted to 4.5 with acetic acid; (c) pH 5.5 phosphate buffer (20 mM) 16.3 g of potassium dihydrogen phosphate in 6 L of water. pH adjusted to 5.5 with NaOH; (d) pH 6.8 phosphate buffer (20 mM) 16.3 g of potassium dihydrogen phosphate in 6 L of water. pH adjusted 6.8 with NaOH. A standard dissolution protocol was used. The standard solution (0.006 mg/mL of eszopiclone free base) was prepared by weighing a known amount (−20 mg) of the free base into a 100 mL volumetric flask. 10 mL of acetonitrile was added into the flask and sonicated until the solids dissolved completely. The solution was cooled to room temperature and made up to the 100 mL mark using media and mixed well. Dilute 3 mL to 100 mL using media. The tables were prepared by transferring 500 mL dissolution media into the 6 vessels. Blank readings were taken for all the probes using media. Standards were read for individual vessels. Probes were washed with media and then inserted back into each vessel. One tablet was transferred into each vessel. The dissolution program was started immediately and readings were taken at 305 nm for 60 min (every 1 min for 20 min and then every 10 min)

The following tables display results of dissolution experiments in deionized (DI) water of uncoated tablets formed from 3 mg free base IR eszopiclone (with no more than 0.3% (R)-zopiclone) with various acids. Average (Avg) reported is the mean % dissolved for the number of tablets used in the dissolution run. n is the number of tablets used in the dissolution run. % RSD is the relative standard deviation from the observed mean. The pH values of the media was measured using a pH paper, in-situ at various time points during the dissolution experiment run in deionized (DI) water of uncoated tablets formed from 3 mg free base IR eszopiclone with 12% L-malic acid. The pH was 6 at 10 min, 20 min., 30 min., 40 min, 50 min., and 60 min

TABLE 1
Dissolution data of uncoated tablets formed from 3 mg free base
eszopiclone and various amounts of fumaric acid.

| | 1% Fumaric, Lot | | 3% Fumaric, Lot | | 9% Fumaric, Lot | | 20% Fumaric, Lot | |
|---|---|---|---|---|---|---|---|---|
| | 2702-30 | | 2702-28 | | 2702-29 | | 2702-31 | |
| Time in min | Avg n = 6 | % RSD | Avg n = 3 | % RSD | Avg n = 4 | % RSD | Avg n = 3 | % RSD |
| 0 | 2 | 192.64 | 6 | 14.75 | 8 | 63.86 | 1 | 249.69 |
| 1 | 10 | 24.41 | 14 | 15.91 | 21 | 20.37 | 19 | 35.03 |
| 2 | 13 | 31.98 | 22 | 20.71 | 29 | 18.29 | 27 | 25.83 |
| 3 | 18 | 22.99 | 27 | 9.39 | 36 | 18.19 | 33 | 23.51 |
| 4 | 21 | 24.43 | 32 | 16.89 | 41 | 11.68 | 39 | 19.46 |
| 5 | 25 | 22.43 | 36 | 10.60 | 46 | 14.00 | 42 | 19.97 |
| 6 | 28 | 23.43 | 40 | 11.91 | 48 | 12.73 | 50 | 25.71 |
| 7 | 30 | 24.25 | 43 | 11.03 | 51 | 13.60 | | |
| 8 | 32 | 18.70 | 48 | 15.22 | 53 | 11.21 | | |
| 9 | 37 | 20.94 | 53 | 22.33 | 55 | 12.38 | 60 | 17.81 |
| 10 | 41 | 22.55 | 52 | 14.59 | 57 | 10.11 | 63 | 16.12 |
| 11 | 41 | 18.64 | 56 | 15.66 | 59 | 10.16 | 64 | 17.37 |
| 12 | 43 | 19.09 | 57 | 14.24 | 61 | 10.50 | 65 | 15.94 |
| 13 | 46 | 16.69 | 59 | 13.04 | 62 | 8.86 | | |
| 14 | 47 | 18.05 | 61 | 12.50 | 64 | 9.16 | | |
| 15 | 49 | 17.74 | 62 | 12.82 | 66 | 8.75 | 70 | 16.83 |
| 16 | 51 | 15.14 | 64 | 10.88 | 67 | 8.52 | 70 | 18.92 |
| 17 | 52 | 15.25 | 66 | 15.26 | 69 | 9.22 | 72 | 16.04 |
| 18 | 54 | 15.46 | 67 | 10.40 | 71 | 6.39 | 73 | 16.04 |
| 19 | 54 | 15.33 | 68 | 9.63 | 71 | 6.34 | 74 | 15.80 |
| 20 | 56 | 14.74 | 69 | 10.67 | 73 | 5.65 | 74 | 15.59 |
| 30 | 64 | 11.89 | 77 | 6.33 | 82 | 4.64 | 83 | 12.22 |
| 40 | 68 | 9.91 | 82 | 3.83 | 88 | 2.98 | 88 | 9.65 |
| 50 | 72 | 9.86 | 86 | 3.91 | 91 | 2.24 | 91 | 8.13 |
| 60 | 73 | 9.61 | 88 | 2.29 | 94 | 2.84 | 94 | 6.02 |

TABLE 2
Dissolution data of uncoated tablets formed from 3 mg free base
eszopiclone and 3% L-malic acid, 12% L-malic acid or 9% maleic acid.

| | 3% L-Malic | | 12% L-Malic | | 9% Maleic | |
|---|---|---|---|---|---|---|
| Time in | Lot 2702-32 | | Lot 2702-33 | | Lot 2702-34 | |
| min | Avg n = 3 | % RSD | Avg n = 6 | % RSD | Avg n = 6 | % RSD |
| 0 | 1 | 46.84 | 1 | 55.63 | 1 | 147.29 |
| 1 | 12 | 16.04 | 8 | 10.47 | 5 | 28.79 |
| 2 | 34 | 6.66 | 18 | 10.91 | 11 | 16.73 |
| 3 | 46 | 9.43 | 26 | 14.34 | 19 | 18.97 |
| 4 | 49 | 7.44 | 37 | 10.16 | 28 | 17.52 |
| 5 | 53 | 11.03 | 47 | 10.71 | 36 | 18.69 |
| 6 | 55 | 13.63 | 57 | 10.99 | 48 | 17.21 |
| 7 | 57 | 9.64 | 66 | 9.17 | 57 | 12.10 |
| 8 | 60 | 7.06 | 72 | 8.51 | 61 | 9.82 |
| 9 | 64 | 8.17 | 75 | 6.95 | 61 | 7.74 |
| 10 | 65 | 6.26 | 75 | 7.55 | 65 | 10.20 |
| 11 | 65 | 6.66 | 77 | 6.97 | 67 | 9.58 |
| 12 | 67 | 6.40 | 77 | 7.01 | 69 | 8.60 |
| 13 | 69 | 4.52 | 76 | 6.25 | 71 | 9.42 |
| 14 | 69 | 5.35 | 77 | 6.11 | 71 | 7.78 |
| 15 | 69 | 5.84 | 78 | 6.80 | 74 | 9.57 |
| 16 | 71 | 2.05 | 79 | 6.94 | 75 | 7.60 |
| 17 | 72 | 3.99 | 78 | 8.59 | 75 | 8.03 |
| 18 | 73 | 4.34 | 78 | 7.14 | 77 | 7.58 |
| 19 | 74 | 4.11 | 78 | 6.20 | 78 | 7.89 |
| 20 | 73 | 5.19 | 80 | 6.45 | 79 | 7.25 |
| 30 | 83 | 2.12 | 84 | 4.97 | 86 | 6.77 |
| 40 | 89 | 3.17 | 87 | 3.98 | 92 | 8.35 |
| 50 | 91 | 2.58 | 89 | 4.32 | 95 | 6.93 |
| 60 | 93 | 1.73 | 91 | 4.47 | 99 | 5.59 |

TABLE 3
Dissolution data of uncoated tablets formed from 3 mg free base
eszopiclone and 9% fumaric acid at pH 1, pH 4.5, and pH 6.8.

| Time | pH 1 | | pH 4.5 | | pH 6.8 | |
|---|---|---|---|---|---|---|
| in min | Avg n = 3 | % RSD | Avg n = 4 | % RSD | Avg n = 4 | % RSD |
| 0 | 3 | 117.22 | 5 | 89.68 | 8 | 43.33 |
| 1 | 36 | 11.83 | 31 | 4.15 | 22 | 26.07 |
| 2 | 46 | 12.63 | 42 | 2.40 | 27 | 8.11 |
| 3 | 58 | 20.74 | 46 | 0.99 | 36 | 12.62 |
| 4 | 62 | 9.99 | 50 | 2.57 | 38 | 8.56 |
| 5 | 68 | 8.44 | 54 | 1.33 | 42 | 12.65 |
| 6 | 72 | 6.72 | 55 | 2.01 | 46 | 7.39 |
| 7 | 77 | 6.38 | 58 | 2.39 | 48 | 5.52 |
| 8 | 81 | 5.01 | 60 | 2.03 | 50 | 4.78 |
| 9 | 82 | 3.32 | 61 | 4.28 | 52 | 4.17 |
| 10 | 85 | 3.74 | 62 | 2.17 | 56 | 5.35 |
| 11 | 88 | 1.17 | 64 | 3.79 | 59 | 7.83 |
| 12 | 88 | 3.21 | 66 | 5.56 | 59 | 5.20 |
| 13 | 89 | 3.23 | 67 | 3.55 | 60 | 6.43 |
| 14 | 89 | 3.26 | 69 | 0.87 | 62 | 5.66 |
| 15 | 90 | 2.69 | 68 | 3.47 | 62 | 5.75 |
| 16 | 90 | 2.67 | 72 | 2.66 | 65 | 2.55 |
| 17 | 93 | 0.41 | 72 | 2.35 | 64 | 5.56 |
| 18 | 92 | 2.58 | 72 | 2.69 | 68 | 7.72 |
| 19 | 90 | 1.36 | 74 | 3.65 | 67 | 5.07 |
| 20 | 92 | 2.13 | 76 | 2.56 | 68 | 5.23 |
| 30 | 94 | 3.23 | 85 | 2.32 | 73 | 1.82 |
| 40 | 94 | 1.64 | 90 | 1.31 | 80 | 4.58 |
| 50 | 93 | 1.14 | 91 | 5.47 | 83 | 5.03 |
| 60 | 95 | 0.58 | 95 | 1.77 | 91 | 6.98 |

We claim:

1. An oral pharmaceutical dosage form comprising:
an effective amount of free base of racemic or enantioenriched zopiclone; and
a super-stoichiometric amount of a solid acid.

2. The oral pharmaceutical dosage form of claim 1, wherein the acid is selected from the group consisting of adipic acid, aspartic acid, glucoheptonic acid, gluconic acid, glutamic acid, lactic acid, mandelic acid, malic acid, maleic acid, fumaric acid, citric acid, ascorbic acid, phosphoric acid, tartaric acid, toluenesulfonic acid, benzenesulfonic acid, succinic acid, monosodium phosphate and glucuronic acid.

3. An oral dosage form according to claim 1 additionally comprising an excipient.

4. An oral dosage form according to claim 1 wherein said acid is present in an amount between about 0.5% by weight and about 30% by weight of said dosage form.

5. An oral dosage form according to claim 1 wherein said acid is present in an amount between about 1% by weight and about 20% by weight of said dosage form.

6. An oral dosage form according to claim 1 wherein said acid is present in an amount between about 2% by weight and about 10% by weight of said dosage form.

7. An oral dosage form according to claim 1 wherein said acid is chosen from malic acid, maleic acid, fumaric acid, citric acid, ascorbic acid, phosphoric acid and tartaric acid.

8. An oral dosage form according to claim 1 wherein said acid is chosen from maleic acid, fumaric acid, L-malic acid and D-malic acid.

9. An oral dosage form according to claim 1 wherein said racemic or enantioenriched zopiclone is present in an amount between about 0.5 mg and about 5 mg.

10. An oral dosage form according to claim 8 wherein said enantioenriched zopiclone is eszopiclone.

11. An oral dosage form according to claim 10 wherein said oral dosage form is a 100 mg tablet comprising between about 0.5 mg to about 3 mg of eszopiclone and between about 0.5 mg to about 20 mg of the acid.

12. A method for rendering the dissolution of orally administered racemic or enantioenriched zopiclone pH independent comprising orally administering racemic or enantioenriched zopiclone free base in the presence of a stoichiometric excess of an acid selected from the group consisting of adipic acid, aspartic acid, glucoheptonic acid, gluconic acid, glutamic acid, lactic acid, mandelic acid, malic acid, maleic acid, fumaric acid, citric acid, ascorbic acid, phosphoric acid, tartaric acid, toluenesulfonic acid, benzenesulfonic acid, succinic acid, monosodium phosphate and glucuronic acid.

13. A method for rendering the dissolution of orally administered racemic or enantioenriched zopiclone pH independent comprising orally administering an oral dosage form according to claim 1.

14. A method for accelerating the postprandial dissolution of orally administered racemic or enantioenriched zopiclone comprising orally administering racemic or enantioenriched zopiclone free base in the presence of a stoichiometric excess of an acid chosen from: adipic acid, aspartic acid, glucoheptonic acid, gluconic acid, glutamic acid, lactic acid, mandelic acid, malic acid, maleic acid, fumaric acid, citric acid, ascorbic acid, phosphoric acid, tartaric acid, toluenesulfonic acid, benzenesulfonic acid, succinic acid, monosodium phosphate and glucuronic acid.

15. A method for accelerating the postprandial dissolution of orally administered racemic or enantioenriched zopiclone comprising orally administering an oral dosage form according to claim 1.

16. A method for improving the bioavailability of orally administered racemic or enantioenriched zopiclone comprising orally administering racemic or enantioenriched zopiclone free base in the presence of a stoichiometric excess of an acid chosen from: adipic acid, aspartic acid, glucoheptonic acid, gluconic acid, glutamic acid, lactic acid, mandelic acid, malic acid, maleic acid, fumaric acid, citric acid, ascorbic acid, phosphoric acid, tartaric acid, toluenesulfonic acid, benzenesulfonic acid, succinic acid, monosodium phosphate and glucuronic acid.

17. A method for improving the bioavailability of orally administered racemic or enantioenriched zopiclone comprising orally administering an oral dosage form according to claim 1.

18. A method according to claim 12 wherein said enantioenriched zopiclone is eszopiclone.

19. A method according to claim 18 wherein said acid is chosen from maleic acid, fumaric acid, L-malic acid and D-malic acid.

20. Use of an oral pharmaceutical dosage form comprising the free base of racemic or enantioenriched zopiclone and a super-stoichiometric amount of an acid chosen from: adipic acid, aspartic acid, glucoheptonic acid, gluconic acid, glutamic acid, lactic acid, mandelic acid, malic acid, maleic acid, fumaric acid, citric acid, ascorbic acid, phosphoric acid, tartaric acid, toluenesulfonic acid, benzenesulfonic acid, succinic acid, monosodium phosphate and glucuronic acid for the treatment of a sleep disorder.
